(19) United States
(12) Patent Application Publication
(10) Pub. No.: US 2018/0000874 A1
(43) Pub. Date: Jan. 4, 2018

(54) Human disease mitigation with food formulation

(71) Applicants: John Edgar Menear, Santa Cruz, CA (US); Joseph Ramaekers, Aptos, CA (US)

(72) Inventors: John Edgar Menear, Santa Cruz, CA (US); Joseph Ramaekers, Aptos, CA (US)

(73) Assignee: CortControl, Santa Cruz, CA (US)

(21) Appl. No.: 15/731,157

(22) Filed: Apr. 27, 2017

Related U.S. Application Data
(63) Continuation-in-part of application No. 14/738,724, filed on Jun. 12, 2015, now abandoned.

(51) Int. Cl. A61K 35/744; A61K 35/20; A23L 33/105; A23L 33/135; A61K 38/17; A61K 36/06; A61K 35/57; A61K 35/54; A23L 33/00; A61K 31/716; A61K 9/00; A61K 8/99; A61K 8/64; A61Q 19/08; A61K 35/00

(52) U.S. Cl. CPC A61K 35/744; A61Q 19/08; A61K 35/20; A23L 33/105; A23L 33/135; A61K 38/1709; A61K 36/06; A61K 35/57; A61K 35/54; A23L 33/30; A61K 31/716; A61K 9/0053; A61K 8/99; A61K 8/64; A23V 2002/00; A61K 2035/11; A61K 2800/92

(57) ABSTRACT

A method of administering a food containing at least transfer factor and lactic acid generating bacteria to mitigate the symptoms of a specific human disease prior to medical or drug intervention. Another food formulation consists of transfer factor, lactic acid generating bacteria, and glucans in appropriate combinations. Other components may be added. The food, administered correctly, reduces cortisol levels, builds the immune system, and balances the endocrine system. Dosage amounts are adjusted for client weight. The method may be used with other treatment options.

### CROSS-REFERENCE TO RELATED APPLICATIONS

This application is a continuation-in-part of U.S. non-provisional application Ser. No. 14/738,724 filled Jun. 12, 2015, and further claims priority to U.S. Provisional Application 61/998,139 filed Jun. 19, 2014. Both priority documents are hereby incorporated by reference in their entirety.

### FIELD OF THE INVENTION

The invention relates to a method of administering food compositions and formulations comprising at least transfer factor and lactic acid generating bacteria to support treatment for several conditions. In some embodiments, glucans are added to the transfer factor and lactic acid generating bacteria. In particular, the conditions addressed with the formulations include (1) early stage Alzheimers, (2) upper respiratory infections, (3) lower respiratory infections, (4) osteoarthritis, (5) osteopenia, (6) osteoporosis and bone weakness (7) proteolysis, (8) muscle wasting, (9) early stage adrenal fatigue, (10) early stage chronic fatigue syndrome, (11) psychological stress, (12) addiction, (13) cancer (various forms), (14) diarrheal disease, and (15) cognitive deficits due to the brain's ability to utilize glucose, (16) skin aging, (17) hypertension, (18) viral infections, (19) irritable bowel syndrome, (20) storage of abdominal fat, (21) multiple sclerosis, (22) anorexia nervosa, (23) candida, (24) intestinal parasites, (25) suboptimal testosterone levels, (26) AIDS, (27) viral hepatitis, (28) weight gain, or (29) arthropenia (loss of joint cartilage).

### BACKGROUND OF THE INVENTION

Veterinary field experience has shown that a combination of transfer factor and lactic acid generating bacteria is useful to address a variety of pet and livestock ailments. Adding glucans to the combination provides nearly a two-fold synergistic effect.

The combination of (1) transfer factor and lactic acid generating bacteria or (2) transfer factor, lactic acid generating bacteria, and glucans is normally administered as a food for a variety of mammals. However, other forms of administration are acceptable.

Success with animal ailments is being adopted for human ailments. Anecdotal human results have demonstrated the benefit of treating humans with the same medical food used for other mammals.

One important benefit of the employed composition is cortisol reduction, which is described more below. However, cortisol reduction is not the only functional effect of the medical food. No healing mechanisms are proposed, but results indicate that cortisol reduction is not acting alone.

The employed medical food also enhances the immune system, and appears to rebalance the endocrine system. Digestive improvement is also involved, which affects overall mammal health.

This application is not limited by any proposed healing mechanism. The results are based on success with animals, not theory. The inventors know that the composition works. The underlying reasons are not fully understood. However, healing mechanisms for each component are cited below.

Cortisol is found in higher-than-normal levels in diseases ranging from AIDS and Multiple Sclerosis to Alzheimer's and heart disease. Prolonged high levels of cortisol can throw the immune system into chaos and ravage the human body. A growing number of researchers believe that many of the worst, and least-understood, diseases will soon be identified as caused (at least partly) by persistently high cortisol.

Published studies have linked high cortisol to the conditions cited in the FIELD OF THE INVENTION above. The cortisol interaction varies. But high cortisol remains a correlating factor.

The immune system plays a particularly important part in the diseases cited in the FIELD OF THE INVENTION. Low immunity leaves the body susceptible to opportunistic pathogens. In contrast, over-active immunity relates to auto-immune diseases; the body attacks itself.

Digestion is the third leg to protect against the diseases cited in the FIELD OF THE INVENTION. A symbiotic relationship exists. A human body has (nominally) 100 trillion cells, of which 90 trillion are intestinal bacteria. The digestive tract is considered the first line of defense against invaders, and lactic acid bacteria support digestive health.

The veterinary field led the way, and developed hard data on food-based solutions for diseases. Compositions based on (1) transfer factor and lactic acid generating bacteria or (2) transfer factor, lactic acid generating bacteria, and glucans were shown to be highly effective for livestock ad pet sicknesses.

In 2006, a discovery was made that these same food compositions lowered cortisol levels in cattle and other mammals. It was later noted that food utilization improved, skin and fur quality improved, weight gains occurred, joint function improved, muscle mass benefited, and energy levels were better. These observations support the use of the food for the disease categories cited in the FIELD OF THE INVENTION.

Those cited disease categories correspond to symptoms that veterinary science uses to diagnose fundamental problems. They are, in themselves, diseases. But they reflect deeper issues. That's why the compositions have wide application. When the immune system, the digestive tract, the endocrine system, and cortisol levels re-equilibrate, the symptoms have no reason for being.

The experience of livestock is significant because the hormonal mechanism of stress is the same in all mammals. Humans generate the stress hormone, cortisol, in the same way as a goat, pig, cow, horse, or monkey. Basically, the pituitary releases ACTH (adrenocorticotropic hormone). Then ACTH stimulates the adrenal cortex to secrete cortisol.

Immunity enhancement has similarities across species. For example, transfer factor from a cow can be used to improve immune response of a human. And good digestion is important across species.

Cortisol has a positive value in the short term. It energizes the body for a fight or flight situation. But a cortisol excess over a long time is destructive. Human health is improved by achieving baseline cortisol levels that fall within a normal concentration range. For years, medical doctors have said that “most diseases arise from stress”.

For example, a negative effect on bone density due to the cortisol excess exists. By rebalancing cortisol (and associated endocrine functions), bone deposition increases and bone loss decreases. Mechanisms such as the calcium malabsorption, hypercalciuria and hypogonadism may apply. Cortisol can mobilize calcium from our bones, and circulate it back into our blood stream. Hypercortisolism, also called Cushing's syndrome, leads to osteoporosis and fractures in up to the 70% of cases, even in the presence of normal gonadal status.

From the viewpoint of Alzheimer's disease, there is new evidence that cortisol can cause damage to brain cells, especially in the hippocampus. New memories are made in the hippocampus. Cells in the hippocampus also serve as a negative feedback circuit to cortisol production. A vicious cycle ensues. As memories become harder to create, the production of cortisol simultaneously increases, more brain cells are lost, more cortisol is produced, and memories become even harder to create.

In addition, cortisol appears to hamper brain cell ability to utilize glucose.

Cortisol affects the digestive tract by killing the friendly bacteria and flora. If someone has irritable bowel syndrome, there's a good chance that cortisol levels are elevated. Intestinal parasites and Candida are likely to thrive in the presence of high cortisol.

High cortisol affects personal appearance. Elevated cortisol reduces synthesis of hyaluronan and proteoglycans, and contributes to their faster degradation. Hyaluronan and proteoglycans are responsible for hydrating the skin; they attract and hold adequate amounts of moisture. Without them, skin develops a dry inelastic quality.

High cortisol lowers testosterone levels and sperm count in males. Libido is reduced in both sexes. Tiredness and fatigue are accompanying symptoms.

Mechanisms are provided above to explain the effects with examples. However, this application is not limited by any proposed mechanism. The core information is that high cortisol, immune imbalance, and endocrine imbalance correlate to various specific diseases.

Human information can be extrapolated from mammal studies. All mammals have a cortisol producing glandular system that mimics humans. Basically, the pituitary gland secretes ACTH (adrenocorticotropic hormone), which then stimulates the adrenal glands to release cortisol. Success with mammals is transferrable to humans.

Cattle studies have repeatedly demonstrated that feeding a mixture of transfer factor and lactic acid generating bacteria facilitates health. Glucans are often added to synergistically increase the immune response.

Medical technology addresses disease with a variety of treatments. Some of these treatments may have side effects. In contrast, the mixture of (1) transfer factor and lactic acid generating bacteria or (2) transfer factor, lactic acid generating bacteria and glucans is safe. Side effects are actually positive because multiple systems are improved. As symptoms recede, the whole body operates better.

Foods take longer to confer benefits than drugs. Depending on disease severity, food compositions confer benefits in three days to ninety days. In return, transfer factor, lactic acid generating bacteria and glucans are inherently safe.

The consuming public now understands that foods possess more than basic nutrition (protein, carbohydrate, fat, etc.). For example, 95% of consumers agree that “certain foods have health benefits that go beyond basic nutrition and may reduce the risk of disease or other health concerns”. More than 50% of consumers believe that foods can replace the use of drugs.

The Federal Drug Administration acknowledges this trend with “medical foods” and “qualified dietary supplements”.

Medical Foods are supervised and monitored by a doctor, nutritionist, nurse, medical technician or equivalent health care professional. Qualified dietary supplements can be dispensed through wider distribution channels.

A food-based treatment option is needed that mitigates root causes of multiple diseases, and avoids negative side effects. A food based treatment may be used in conjunction with other treatments.

### BRIEF SUMMARY OF THE INVENTION

This instant invention is a method of treating multiple diseases and clinical indications with foods that reduce human cortisol levels, build immunity, and balance endocrine function (among other functional effects). Areas of application include (1) early stage Alzheimers, (2) upper respiratory infections, (3) lower respiratory infections, (4) osteoarthritis, (5) osteopenia, (6) osteoporosis and bone weakness (7) proteolysis, (8) muscle wasting, (9) early stage adrenal fatigue, (10) early stage chronic fatigue syndrome, (11) psychological stress, (12) addiction, (13) cancer (various forms), (14) diarrheal disease, and (15) cognitive deficits due to the brain's ability to utilize glucose, (16) skin aging, (17) hypertension, (18) viral infections, (19) irritable bowel syndrome, (20) storage of abdominal fat, (21) multiple sclerosis, (22) anorexia nervosa, (23) candida, (24) intestinal parasites, (25) suboptimal testosterone levels, (26) AIDS, (27) viral hepatitis, (28) weight gain, or (29) arthropenia (loss of joint cartilage).

Following is a condensed summary of the invention. By necessity, details are omitted in order to simply state the essence of the invention. Omitted details within this section should not be construed in a way that limits or alters the scope of the invention.

One preferred medical food composition is a mixture of transfer factor and lactic acid generating bacteria. This composition is described in U.S. Pat. No. 6,962,718 issued to Joseph Ramaekers.

Another preferred food composition is a mixture of transfer factor, lactic acid generating bacteria, and glucans. Glucans may be present as mushrooms. This composition is encompassed by domineering U.S. Pat. No. 6,962,718, but contains one additional component (glucans).

Either preferred composition may be augmented with additional additives. Those additives may differ depending on the disease condition addressed. But either (1) transfer factor and lactic acid generating bacteria or (2) transfer factor, lactic acid generating bacteria and glucans are present in formulations that fall within the scope of this invention.

Dosages of medical foods are adjusted for patient weight. Heavier patients typically consume higher dosages.

The relative proportion of transfer factor, lactic acid generating bacteria, and/or glucans within a dose may vary. For example, an increase in glucans is appropriate when auto-immune disorders are present. Although typical proportions can be recited, proportions may be modified to best serve each individual.

For some clients, transfer factor, lactic acid generating bacteria, and glucans are taken together. For other clients, transfer factor, lactic acid generating bacteria, and glucans are taken at different times during the day or week. Component separation and consumption of each component at different times are within a two day period are covered by the scope of this invention.

Typical method-of-use steps include some or all of the following: (1) deciding that one of the conditions in the FIELD OF THE INVENTION is an issue, (2) selecting the medical food dosage level based on weight, (3) consuming the food 3-90 days prior to medical or drug intervention, (4) continuing food consumption until symptoms are reduced, (5) measuring cytokine or hormonal levels (for example, cortisol) over the treatment period, and (6) adjusting dosage levels as appropriate.

Consumption for hree to ninety days prior to medical intervention is recommended to allow the recipient's immune system and endocrine system to re-invigorate. The administered composition is a food, not a drug. Response times are longer for a food.

Medical food consumption may be used alone or used with other treatments. If pharmaceutical drugs are employed in a treatment regimen, the health care professional may elect to continue the pharmaceutical drugs along with the food composition.

Transfer of successes from livestock disease to human disease is supported by several facts. First, cortisol biochemistry is the same among mammal species. Second, transfer factor functions the same across species. Third, lactic acid generating bacteria improve digestive health across the mammal spectrum. Forth, both transfer factor and glucans benefit the immune response of most mammals.

The scientific basis of each component is contained in the DETAILED DESCRIPTION below.

Human benefits have been observed, but have been difficult to document. Pet owners have admitted to buying the food for a pet, but then taking it themselves. Results have been positive. Some owners continue to purchase the medical foods long after the pet died.

Informal studies also suggest the connection between the medical food and human performance. Feeding the medical food to stressed athletes lowers the symptoms of cortisol excess. But more than cortisol is involved. With the medical food, athletes tend to get a better night's sleep, and perform better during events. College students consume these foods before final examinations, sleep better, and get better grades. Golfers use these foods to lower scores. Elite military squads are currently evaluating mission recovery.

A cortisol-to-disease correlation in humans and other mammals is supported by a preponderance of data. Lowered cortisol measurements correlate to consuming transfer factor, lactic acid generating bacteria, and/or glucans. Other stress hormones may also be lowered by consuming transfer factor, lactic acid generating bacteria, and/or glucans.

In humans, better skin quality plus improved flexibility and movement are noticeable outcomes.

### BRIEF DESCRIPTION OF THE DRAWINGS

FIG. 1 is a diagram of cortisol generation in mammals. Cortisol is cited as important because it is a primary stress hormone. But cortisol reduction does not fully explain the benefits of the employed medical food.

FIG. 2 shows a drop in cortisol for calves that consumed the medical food, relative to control calves that received a placebo. Cortisol reduction played an important part in reviving their health because the calves were considered stressed from shipment.

FIG. 3 shows an increase in insulin for test calves. Under the test conditions, the increased insulin translates into better food utilization. Food utilization is quantified by calf weight gain (see FIG. 4). Note that the insulin increase began on the seventh consumption day, and the positive effects continued for at least 60 days.

FIG. 4 shows the daily average weight gain of calves receiving medical food. Measurements were made at 12 and 60 days. The weight gain was categorized by veterinary doctors to be largely muscle, bone, and ligaments. This is the opposite of bone loss, muscle wasting, osteopenia, osteoporosis, bone weakness, proteolysis, or arthropenia (loss of joint cartilage).

FIG. 5 shows a drop in morbidity and mortality of cattle for a first herd, which unmistakably signals better health. Improved health is reflected in reduced symptoms for the conditions cited in the FIELD OF THE INVENTION. Morbidity and mortality were largely traceable to respiratory infections, diarrheal disease, hypertension, viral infections, candida, and intestinal parasites.

FIG. 6 shows a drop in morbidity and mortality of cattle for a second herd. Again, results of medical food administration are significant.

FIG. 7 shows a calf with a severe case of warts before composition consumption.

FIG. 8 shows the same calf in FIG. 7 after composition consumption. The warts are gone.

FIG. 9 shows weight gain of pigs in a controlled study. Pigs receiving the composition gained 3.9 pounds more (on average). The weight gain was muscle and bone.

### DETAILED DESCRIPTION OF THE INVENTION

The instant method is designed to use a food to strengthen the immune system, rebalance the endocrine system, reduce cortisol levels, or supply/create needed cytokines. A preferred food is a composition containing transfer factor, glucans, and lactic acid generating bacteria.

The method is a suggested first-tier intervention. Consumption 3-90 days prior to medical intervention is recommended. However, longer or shorter pre-medical consumption periods may be used by a doctor on an individual basis.

Transfer factor is produced by leucocytes and lymphocytes. Transfer factor comprises small water soluble polypeptides of about 44 amino acids that stimulate or transfer cell mediated immunity from one individual to another.

The properties, characteristics and processes for obtaining transfer factor or transfer factors are discussed in U.S. Pat. Nos. 4,816,563; 5,080,895; 5,840,700, 5,883,224 and 6,468,534, the contents of which are hereby incorporated by reference into the present application.

Alternative sources of transfer factor include avian transfer factor, ova transfer factor, and colostrum from goats, pigs, horses and humans. This listing is not complete. In addition, pooled combinations of transfer factors from any number of sources may be used.

In certain embodiments, substantially purified transfer factor has a molecular weight of less than 10,000 Daltons.

Transfer factor is commercially available, and known to be safe.

Fractions within transfer factor serve different (and sometimes opposite) purposes. Two particularly important fractions are (1) the inducer fraction, and (2) the suppressor fraction. Opposing functions were confirmed using the direct Leucocyte Migration Inhibition (LMI) test.

These two fractions are consistent with field observations that transfer factor balances the immune system.

The inducer fraction of transfer factor links the immune cells with an antigen-binding site, thereby increasing their reactivity to an antigenic stimulus. This is advantageous when a higher immune response is required. CortControl's enhanced vaccine response (a separate application) arises from an inducer fraction. The inducer effects of transfer factor are documented by several researchers.

The suppressor fraction blocks the response of the T-cells and signals a down-regulation of the immune response. This is useful in allergic or autoimmune conditions. Animal studies demonstrate improvement for cases where the immune system is over-active. This arises from suppressor fraction. Cortesini, R. et.al. demonstrated that CD8+CD28−Ts represent a unique subset of regulatory cells within transfer factor that initiates a suppressive loop. Filaci supported Cortesini's work, and related auto-immune disease the absence of CD8+ suppressor T lymphocytes. Filaci further found that “CD8+ Ts can be generated in vitro from CD8+CD28−T lymphocytes. A key role in their generation is played by monocytes that secrete interleukin-10 (IL-10) after granulocyte macrophage-colony-stimulating factor (GM-CSF) stimulation.”

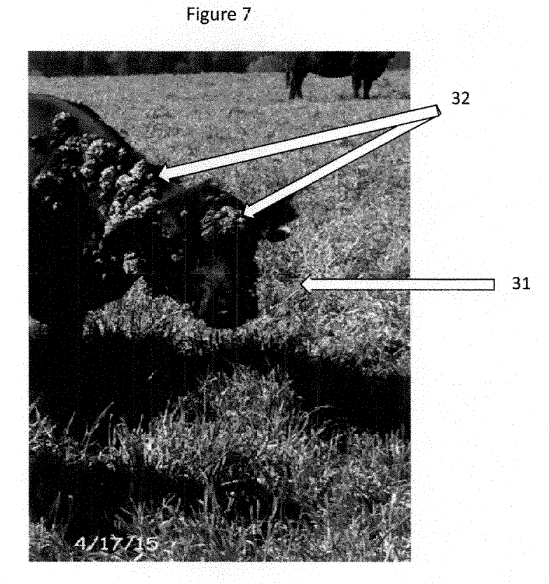

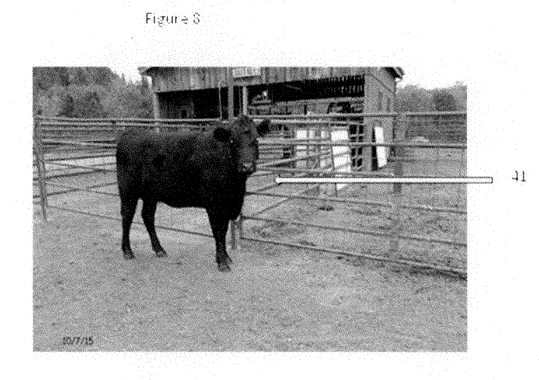

Aleli Salazar-Ramiro et.al' view cancer as an auto-immune disease, and propose transfer factor for cancer therapy. This is supported by CortControl's veterinary work on horses, dogs, and cows, where symptoms of cancer were visibly reversed. This is shown in FIGS. 7 and 8. In FIG. 7, the calf 31 developed severe warts 32 before the food composition was introduced into the diet. FIG. 8 shows the same calf 41. Warts receded after consumption of the food composition.

Transfer factors contain a spectrum of cytokines from both TH1 and TH2 lymphocytes. TH1 and TH2 cells perform different functions and produce different cytokines. Cytokines are proteins that function as messenger molecules, and promote health by balancing the immune system and endocrine systems.

TH1 cells produce the following cytokines: IL-2, IFN-gamma, and TNF-alpha.

TH2 cells modulate humoral immunity (antibody production). TH2 cells produce the following cytokines: IL-4, IL-5, IL-6, IL-10, and IL-13.

Diseases can be categorized as TH1 predominant or TH2 predominant.

If one has a TH2-dominated condition, the disease conditions that tend to prevail are shown in Table 1 below.

TABLE 1

| | |
|---|---|
| 1. | Allergies |
| 2. | Chronic sinusitis |
| 3. | Atopic eczema |
| 4. | Asthma |
| 5. | Systemic autoimmune conditions such |
| | as lupus erythematosus and mercury- |
| | induced autoimmunity |
| 6. | Vaccination-induced state |
| 7. | Certain cases of autism |
| 8. | Hyperinsulinism |
| 9. | Pertussis vaccination |
| 10. | Malaria |
| 11. | Helminth infection |
| 12. | Hepatitis C |
| 13. | Chronic giardiasis |
| 14. | Hypercortisolism |
| 15. | Chronic candidiasis |
| 16. | Cancer |
| 17. | Viral infections |
| 18. | Ulcerative colitis |

If one has a TH1-dominated condition, the conditions that tend to prevail are shown in Table 2 below.

TABLE 2

| | |
|---|---|
| 1. | Diabetes type 1 |
| 2. | Multiple sclerosis |
| 3. | Rheumatoid arthritis |
| 4. | Uveitis |
| 5. | Crohn's disease |
| 6. | Hashimoto's disease |
| 7. | Sjögren's syndrome |
| 8. | Psoriasis |
| 9. | Sarcoidosis |
| 10. | Chronic Lyme disease |
| 11. | H. pylori infections |
| 12. | E. histolytica |

Transfer factor contains cytokines from both TH1 and TH2 cells. And transfer factor produces different cytokines for different situations. Transfer factor selectively affects cytokine production in response to antigenic stimulation.

If a person is predominately TH1, transfer factor balances the immune system toward TH2. If a person is predominately TH2, transfer factor balances the immune system toward TH1. A balanced TH1/TH2 is most desirable.

Transfer factor can change TH1/TH2 predominance conditions within 48 hours. This cannot be due to new helper cells because it takes 10-14 days to mature new helper cells. The capability is provided by eomesodermin, which is present in transfer factor.

Cytokines are functionally similar to hormones, but are not associated with a specific gland. Cytokines appear to operate as a key and template. Stereochemistry is important. Attachment of a cytokine on an immune cell receptor will start a specific immune signal within the target cell. Signals translate into direct action, increase the production of antibodies against an invading virus, or initiate the production of other cytokines to propagate the signal.

Lactic acid generating bacteria is an important component of the pertinent medical foods, and is GRAS (generally recognized as safe). Lactic acid generating bacteria support digestion and brain health. Lactic acid generating bacteria provide healthful effects that are found in non-pasteurized sauer kraut and cod liver oil. Within the intestinal tract, lactic acid generating bacteria are beneficial. It has been estimated that 80% of human health depends on beneficial intestinal bacteria.

A human body becomes stressed by poor digestion. Poor digestion contributes to high cortisol levels, intestinal parasitic buildup, weakened bones, muscle wasting, skin aging, early stage Alzheimers, upper respiratory infections, lower respiratory infections, osteoarthritis, osteopenia, osteoporosis, proteolysis, early stage adrenal fatigue, early stage chronic fatigue syndrome, psychological stress, addiction, cancer (various forms), diarrheal disease, cognitive deficits, hypertension, viral infections, irritable bowel syndrome, storage of abdominal fat, multiple sclerosis, anorexia nervosa, candida, suboptimal testosterone levels, AIDS, viral hepatitis, weight gain, and arthropenia (loss of joint cartilage). Lactic acid generating bacteria help reduce cortisol and increase the immune system via improved digestion.

The gastrointestinal tract is a complex ecosystem with hundreds-to-thousands of microflora and bacteria species. Some species are beneficial, such as lactobacillus or bifidobacteria.

The gastrointestinal tract has the dual role of excluding pathogens while facilitating the absorption of nutrients. That's not easy because gut-associated tissue is the largest lymphoid tissue of the human body. Of particular importance is failure to exclude pathogens (leaky gut). This condition allows pathogens to access the whole body.

There is a connection between the gastrointestinal tract, the overall immune system, and lactic acid bacteria presence. Disease and destruction appear when pathogens overwhelm the body's natural defenses. This is doubly important when needed nutrition is blocked by inadequate absorption. Muscle loss (arthropenia) and bone loss (osteopenia) are worsened by poor nutrient absorption.

Food and water are common sources of pathogens, particularly in less developed countries. That's why intestinal health is considered the first line of defense. That line of defense is important to avoid a spectrum of diseases. If the entire immune system is weakened by intestinal problems, the ability to defend against other issues is inadequate.

Intestinal microbiota are considered an adaptable and rapidly renewable organ of the body. Ecosystem composition can be modified. Diet is one modifier. Species that receive their preferred nutrients will flourish. Species that don't are disadvantaged in a Darwinian competition. Probiotic supplements are a second modifier. Lactic acid generating bacteria are preferred components.

The importance of balanced lactic acid generating bacteria on good digestion is understood. But digestion is not the only benefit. The ecosystem within the GI tract is responsible for other essential processes including: immune system regulation, vitamin bioavailability, sleep cycle, brain health, cytokine production, and vitamin K production.

Lactic acid generating bacteria compete against or “antagonize” an array of pathogens including Escherichia coli (E. coli, the cause of “Montezuma's Revenge”), Staphylococcus aureus and Salmonella (common causes of food poisoning), Candida albicans (yeast infections and syndromes), and other pathogens such as Shigella, Clostridium, Listeria, and Helicobacter species.

Claimed benefits of lactic acid generating bacteria include improved immune health, improved mood and mental health, higher energy levels, regulation of hormone levels, reduced yeast infections, and weight control. Success is reported for treatment of diarrhea, Irritable Bowel Syndrome, Crohn's disease, gut dysbiosis, vaginal yeast infections, and urinary tract infections. Lactic acid generating bacteria produces lactase, which helps people with lactose intolerance. Advantageous effects on brain health, immune support, allergies, fertility, cytokine and chemokine production, vaccine enhancement, and Th1/Th2 status are discussed below.

A healthy intestinal lining has two functional properties. Both are enhanced by lactic acid generating bacteria.

The first is a mechanical barrier. Bacteria lining the intestines reinforce the barrier that prevents pathogens from seeping out of the GI tract and into the body. This is particularly important in the large intestine, where fecal matter accumulates before being excreted. Without a strong barrier, toxins from feces can be reabsorbed, placing an additional burden on the immune system.

The second feature is a thick mucus coating that lines the intestines. Lactobacillus and Bifidobacterium are important because they produce butyric acid, which is essential to growing the thick mucus coating. Pathogens become trapped in the mucus. Then the mucus sloughs off and carries pathogens with it, leading to faster elimination.

Micro-species exist in equilibrium. Supplementing with lactic acid generating bacteria shifts the equilibrium toward a healthy bacteria balance. The improved equilibrium sets up a positive feedback loop as lactic acid generating bacteria secretions further weaken the remaining pathogens.

Cytokines and chemokines are produced inside the intestines. Intestinal epithelial cells respond to certain bacteria by producing an array of cytokines and chemokines which support host immunity. Various experiments indicate that the ability to secrete various cytokines is mediated to a large extent by cell wall components.

Glucans (polysaccharides) are known to support the immune system. Animals pretreated with purified glucan particles are subsequently more resistant to bacterial, viral, fungal, and protozoan challenge, reject antigenically incompatible grafts more rapidly and produce higher titers of serum antibodies to specific antigens.

An important function of beta-glucans is immuno-modulation. A century and a half of research has shown that beta glucans act as immuno-modulating agents, meaning they trigger a cascade of events that help regulate the immune system, making it more efficient.

Beta glucans effectively bind and activate specific innate immune cells including T-cells, NK (natural killer) cells, and macrophages. The ability of beta glucans to modify the attack of immune cells on invasive agents supports an efficient and stronger immune response, while causing minimal damage to the rest of the body. “Minimal damage” includes avoidance of over-stimulation, which leads to autoimmune diseases.

Immuno-suppression is observed in people with stress-related diseases, such as coronary disease. Under stress, the number of macrophages available are reduced and unable to participate in the immune cascade. This causes even deeper immuno-suppression. Beta-1,3/1,6-glucan has been shown to nutritionally potentiate and activate macrophage cells which may assist in countering these effects.

Beta-glucans provide value in 6 areas: (1) boost the immune response, (2) activate and modify macrophage response, (3) signal via cytokines, (4) provide response specificity, (5) prevent over-stimulation, and (6) opsinize viral, bacterial, fungal, and parasitic invaders.

In order to function optimally, macrophages must pass through a state of activation which involves certain form and structure changes. Those changes are effected when a human (or mammal) ingests mushrooms with a potent level of beta-glucans.

The boost in response due to beta-glucans begins with macrophage interaction. Macrophages (“big eaters”) ingest the beta-glucan particles, and become activated in the process. An activated macrophage sends signals (cytokines) to produce more macrophages in the bone marrow.

CR1, CR3, TLR-2/6 and Dectin-1 receptors on the macrophage bind to the beta-glucans. Of these, Dectin-1 has received increasing study, and is cited as a major beta-glucan receptor in macrophages, neutrophils, and dendritic cells. Dectin-1 is a type II transmembrane protein receptor that binds β-1,3 and β-1,6 glucans. (SIGNR1) is a major mannose receptor on macrophages that cooperates with Dectin-1.

Dectin-1 is expressed on the surface of all macrophage populations tested as well as on monocytes, dendritic cells, and neutrophils, demonstrating that Dectin-1 is not restricted to cells of the dendritic cell lineage.

Beta-glucans are not highly soluble, and particles that pass through the intestines are relatively large. Enterocytes facilitate the transportation of β(1,3)-glucans and similar compounds across the intestinal wall into the lymph, where they begin to interact with macrophages to activate immune function. Large beta-glucans are broken down in the macrophage, and made available to other immune cells.

Cheung-VKN et al. labeled β-glucans with fluorescein to track their oral uptake and processing in vivo. The orally administered β-glucans were taken up by macrophages via the Dectin-1 receptor and were subsequently transported to the spleen, lymph nodes, and bone marrow. Within the bone marrow, macrophages degraded the large β-1,3-glucans into smaller soluble β-1,3-glucan fragments. These fragments were subsequently taken up via the complement receptor 3 (CR3) of marginated granulocytes. These granulocytes with CR3-bound β-glucan-fluorescein were shown to kill inactivated complement 3b (iC3b)-opsonized tumor cells after they were recruited to a site of complement activation.

Beta-glucan action is mediated via the activated complement receptor 3 (CR3, also known as CD11b/CD18), which is found on natural killer (NK) cells, neutrophils, and lymphocytes. This pathway is responsible for opsonic recognition of β-glucans leading to phagocytosis and reactor cell lysis. Beta-glucans bind to the lectin domain of CR3 and prime it for binding to inactivated complement 3b (iC3b) on the surface of reactor cells.

Two other receptors known as scavenger and lactosylceramide also bind β-glucans and can elicit a range of responses. β-glucans can enhance endotoxin clearance via scavenger receptors by decreasing TNF production.

β-glucans binding to lactosylceramide receptor can enhance myeloid progenitor proliferation and neutrophil oxidative burst response, leading to an increase in leukocyte anti-microbial activity. It is also associated with the activation of NF-κB in human neutrophils.

Orally administered yeast-glucan was reported to decrease the levels of IL-4 and IL-5 cytokines responsible for the clinical manifestation of allergic rhinitis, while increasing the levels of IL-12. Cytokine release results in a coordinated attack.

A signaling cascade arises when immune receptors interact with β-1,3 and β-1,6 glucans. Several signaling molecules have been reported to be involved. They are NF-κB (through Syk-mediate pathway), signaling adaptor protein CARD9 and nuclear factor of activated T cells (NFAT). This will eventually lead to the release of cytokines including interleukin (IL)-12, IL-6, tumor necrosis factor (TNF)-α, and IL-10.

Beta-glucans balance the immune response, and prevent over-stimulation. More immune response isn't always better.

Decreased immunity results when the immune system is over-stimulated with real and perceived threats. With time, this leads to a weakened immune system, and the individual becomes more susceptible to development of cancer and infections. Individuals with over-stimulated immune systems have an increased risk of chronic inflammatory conditions such as autoimmune disorders and asthma. Complement receptors are related to glucan modulation.

For example, (1) Membrane cofactor protein is a widely distributed C3b/C4b binding regulatory glycoprotein of the complement system; (2) Decay-accelerating factor (DAF: CD55: Cromer antigen) protects host cells from complement-mediated damage by regulating the activation of C3 convertases on host cell surfaces; and (3) Complement receptor 2 is the C3d receptor.

Immune specificity to fight viruses, fungi, bacteria, and parasites arises from the following facts: (1) viruses, fungi, and bacteria contain beta-glucans, (2) healthy humans do not manufacture beta-glucans, (3) a macrophage that has been trained to detect beta-glucans will eliminate viruses, fungi and bacteria without attacking human cells, and (4) in a summary statement, “self” is distinguished from “invader”. The immune response is specific, and doesn't harm the host.

Beta-glucan is an opsonin. An opsonin is any molecule that enhances phagocytosis by marking an antigen for an immune attack or marking dead cells for recycling.

Opsonization (also, opsonisation) is the molecular mechanism whereby molecules, microbes, or apoptotic cells are chemically modified to have stronger interactions with—to be more “delicious” to cell surface receptors on phagocytes and NK cells. With the antigen coated in opsonins, binding to immune cells is greatly enhanced.

A coating of beta-glucans on an invader makes the invader more susceptible to leukocyte removal. Binding reaction is greatly enhanced. Opsonin translates as “to prepare for eating”. Opsonization further mediates phagocytosis via signal cascades from cell surface receptors.

Opsonins aid the immune system in a number of ways. In a healthy individual, they mark dead and dying self cells for clearance by macrophages and neutrophils, activate complement proteins, and target problems for destruction through the action of natural killer (NK) cells.

Although transfer factor, glucans and lactic acid generating bacteria produce immunity efficiency, they do it differently. Each component plays a unique part in immunity, and addresses another aspect of the immune system. Combining the components doesn't simply create an additive effect. Instead, the combination creates a broadening effect by addressing individual aspects of the immune system plus interactive effects.

For example, beta glucans are opsonins that activate macrophages. Neither transfer factor nor lactic acid generating bacteria can perform that function. That activation pathway requires CR1, CR3, TLR-2/6 and Dectin-1 as attachment sites. Neither transfer factor nor lactic acid generating bacteria operate through CR1, CR3, TLR-2/6 and Dectin-1.

In another example, only transfer factor directly supplies a broad mixture of cytokines, available for immediate use. The transfer factor itself contains a concentrated source of cytokines.

Glucans and lactic acid generating bacteria interact with the pathogens and macrophages to create specific cytokines. But cytokines are not immediately provided, and the cytokine profile is linked to pathogens and macrophages.

Further, enterocytes are required to facilitate the transportation of β(1,3)-glucans (due to large size) across the intestinal wall into the lymph, where they begin to interact with macrophages to activate immune function. No similar pathway is required by lactic acid generating bacteria and transfer factor.

The combination of transfer factor, glucans and lactic acid generating bacteria performs functionally and biochemically different than a person of ordinary skill would expect. Although they all address similar issues, an attempt to view them as the addition of three similar species would lead to an incorrect model.

When glucans are combined with transfer factor and lactic acid generating bacteria, an unexpected interaction is created. The combined effect on health is greater than the effect predicted from summing the individual components. That synergy is dramatic—approximately two-fold.

Once a disease is diagnosed, the treatment should address the underlying causes. Elevated cortisol, compromised immunity, and imbalanced endocrine function are often among the underlying causes.

Most likely medicines will be prescribed, but medicines can have undesirable side effects.

A preferred food composition is a mixture of transfer factor, lactic acid generating bacteria, and glucans. Glucans may be present as mushrooms. The method of using transfer factor, lactic acid generating bacteria, and glucans for treating specific diseases can also be viewed as an improvement to U.S. Pat. No. 6,962,718.

Addition of glucans to transfer factor and lactic acid generating bacteria creates a measureable immunity synergy that was not expected. Field tests showed that transfer factor plus lactic acid generating bacteria increased killer T cells by 250%, and glucans increased killer T cells by 40%. But the combination of transfer factor, lactic acid generating bacteria and glucans increased killer T cells by 500%.

The composition components—(1) transfer factor and lactic acid generating bacteria or (2) transfer factor, lactic acid generating bacteria and (3) glucans—may be augmented with additional additives. Chosen additives vary with the intended method of use. Example additives are minerals, probiotics, prebiotics, dimethyl glycine, ascorbic acid, Vitamin A, Vitamin D3, Vitamin E, Vitamin B1, Vitamin B2, Vitamin B12, dipotassium phosphate, potassium chloride, magnesium sulfate, calcium pantothenate, minerals, antioxidants, amino acids, nutraceuticals, inositol hexaphosphate (Ip6), mannans, olive leaf extract, and phytosterols. In certain preferred embodiments, mannans are derived from Aloe vera. In certain preferred embodiments, phytosterols may be derived from soya bean.

Probiotics additives include, but are not limited to B. subtlis, B. longum, B. thermophilium, B. coagulans, E. faecium, and S. cerevisia, L. casei, L. plantarum, Pediococccus acidilacticii, Kluyveromyces marxianus fragillis and combinations thereof.

The above listings do not include all possible additives. The food compositions may also include one or more of the following: carrier proteins such as serum albumin; buffers such as sodium acetate; fillers such as microcrystalline cellulose, lactose, corn and other starches; binding agents; sweeteners and other flavoring agents; coloring agents; and polyethylene glycol. Additives are well known in the art, and are used in a variety of formulations.

The relative proportion of transfer factor, lactic acid generating bacteria, and glucans within the composition may vary widely.

However, some reasonable weight ranges for transfer factor are 0.05-50 mg/pound of body weight. Reasonable weight ranges for lactic acid generating bacteria are 0.47-10 mg/pound of body weight. This is based on a nominal live count of 2.5×10⁹ CFU/Ounce. Reasonable weight ranges for glucans are 0.1-10 mg/pound of body weight.

The method of using the composition may have some or all of the following steps:
(1) determine with measurement that cortisol, immune function, or endocrine balance is a correlating factor to a human's specific disease,
(2) select the correct proportion of transfer factor, lactic acid generating bacteria, and glucans,
(3) choose the correct dosage level, based on human weight,
(4) select a feeding frequency between five times per day and once per week,
(5) begin consumption prior to standard drug or medical treatment,
(6) periodically adjust dosage,
(7) continue consumption until a positive outcome is achieved, and
(8) continue consumption until a drug treatment begins.

A preponderance of test data supports the effect of administering transfer factor, lactic acid generating bacteria, and/or glucans to overcome livestock diseases. Animal data is not pursued in depth herein because this instant application is focused on human diseases. However, humans are mammals, and livestock data is useful.

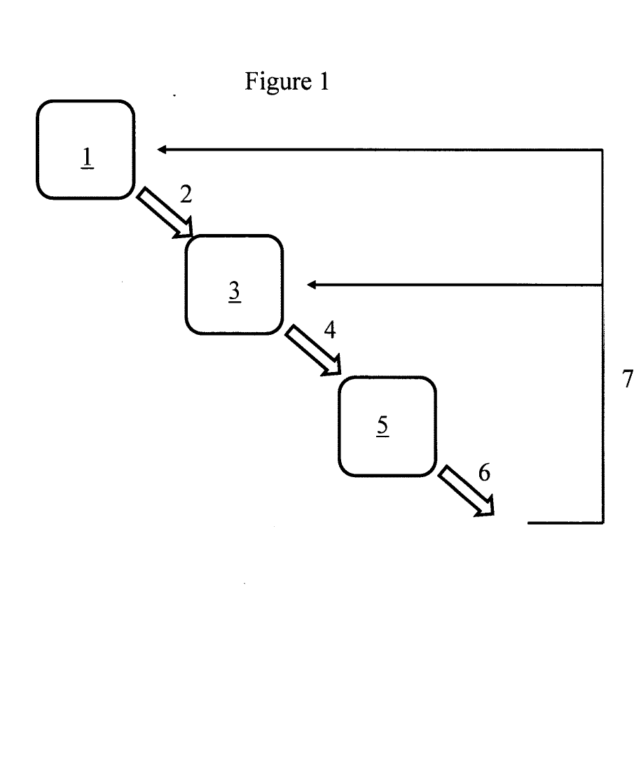

FIG. 1 shows the cortisol production sequence. FIG. 1 applies to humans and other mammals. The hypothalamus 1 releases corticotropin releasing hormone 2, which causes the anterior pituitary gland 3 to secrete adrenocorticotropic hormone 4. Adrenocorticotropic hormone 4 travels to the adrenal cortex 5, which responds by producing cortisol 6. Levels of cortisol are controlled by negative feedback loops 7.

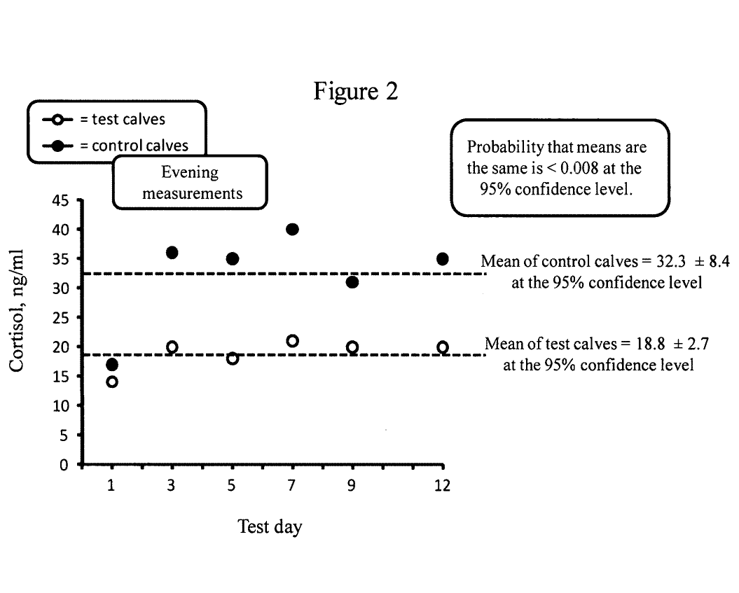

FIG. 2 graphically shows a drop in evening cortisol for stressed calves when fed a mixture of transfer factor, lactic acid generating bacteria, and/or glucans. This graph is provided to show how cortisol is decreased in mammals by feeding the appropriate medical food.

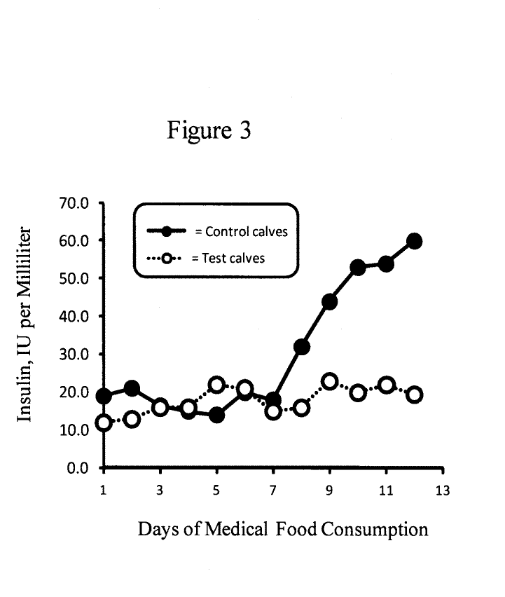

FIG. 3 shows how medical food affected insulin levels in cattle when an increase was desired. This shows that improvements are not limited to cortisol reduction alone. Other hormones are affected. As the calves became healthier, food utilization improved. This was detected as an insulin level increase. Note that it required 7 days to see this effect, and the effect continued beyond 12 days.

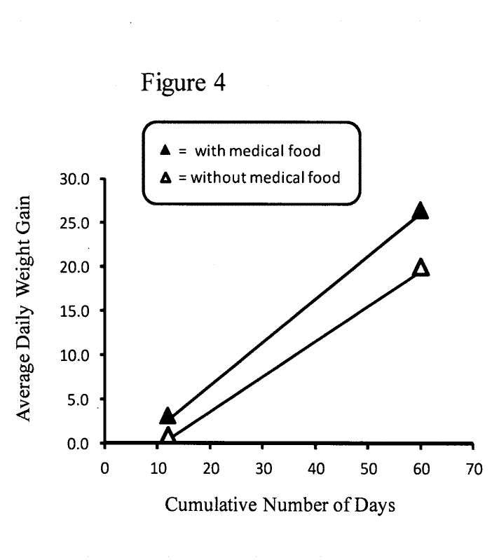

FIG. 4 is associated with FIG. 3. FIG. 4 graphically shows daily calf weight gain over a 60 day period. The weight gain correlated with the increased insulin levels in FIG. 3 and reflects more efficient feed conversion.

Calf weight gain supports the application for soft tissue and bone health. Veterinary observation indicated that the weight gain was largely from bone, muscle, and ligaments. Any hints of osteoarthritis, osteopenia, osteoporosis, arthropenia, muscle wasting, or bone weakness were being reversed. Skin quality and fur appearance were noticeably better.

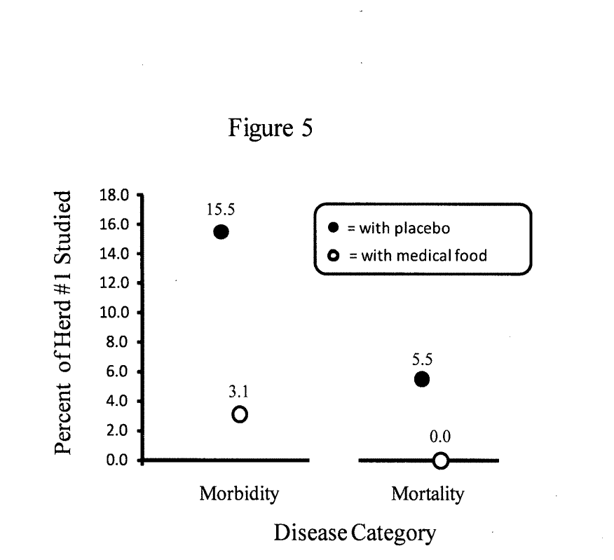

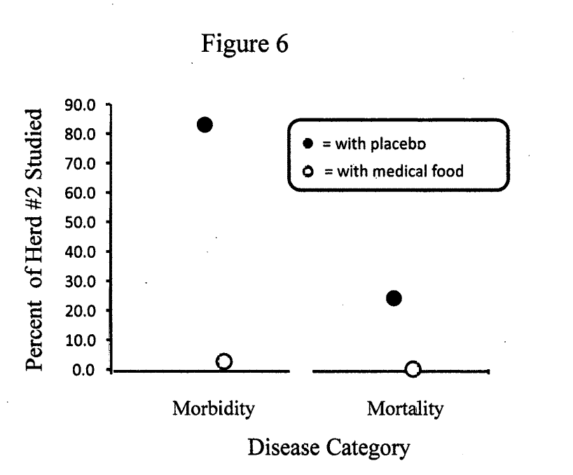

FIG. 5 and FIG. 6 show a drop in both morbidity and mortality for two different infected cattle herds. In both cases, viral and parasitic infections were involved. Respiration was affected. Diarrhea was common. Antibiotics were tried, but the herds remained sick. In contrast, the food solved the problem. This demonstrates the composition's value for upper respiratory infections, lower respiratory infections, diarrheal disease, viral infections, irritable bowel syndrome, candida, intestinal parasites, AIDS, and viral hepatitis.

Performance data substantiate the composition's value for early stage adrenal fatigue, early stage chronic fatigue syndrome, psychological stress, addiction, and hypertension. For example, race horses show marked improvement when the composition is added to the diet. Unpublished CortControl research shows special military forces recover faster from stressful missions. Golfers shoot lower scores after consumption. Verbal reports describe how improved performance evolves from a less stressed mindset.

Several stress hormones and chemical markers benefit from a mixture of transfer factor, lactic acid generating bacteria, and/or glucans. Examples include alpha amalyase and T4 measurements of thyroid function.

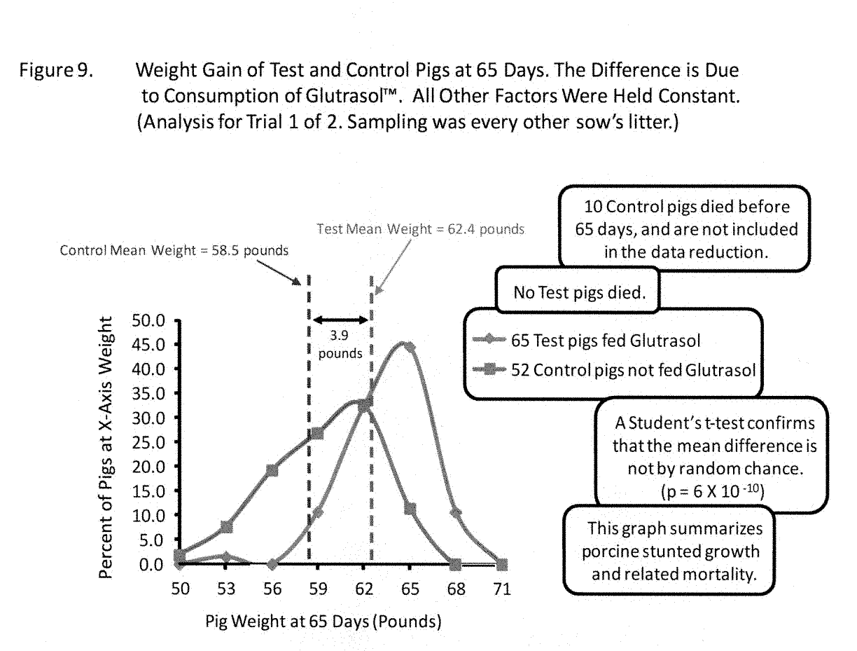

FIG. 9 shows an average weight gain of 3.9 pounds for pigs receiving the composition. When the pigs were processed, the weight gain was attributed to bone and muscle. This data demonstrates value for osteoporosis, bone weakness, proteolysis, and muscle wasting.

Again, this application is based on actual observations—not theory. Although chemical markers and mechanisms are discussed, the scope of this application is not limited by and proposed mechanisms.

The invention claimed is:

1. A method for reducing the symptoms of human disease, comprising:
combining transfer factor and lactic acid generating bacteria to create a food, wherein
said transfer factor includes polypeptides with a molecular weight below 10,000 Daltons, and
said transfer factor is derived from avian, ova or colostrum sources;
feeding the food to said human, wherein
a dosage level is chosen based on the human's weight,
feeding begins 3-90 days before medical or drug intervention, and
the frequency of the feeding ranges between five times per day and once per week; and
continuing said feeding until medical or drug intervention begins.

2. The method of claim 1 wherein said human disease includes any one selected from a group consisting of (a) early stage Alzheimers, (b) upper respiratory infections, (c) lower respiratory infections, (d) osteoarthritis, (e) osteopenia, (f) osteoporosis and bone weakness (g) proteolysis, (h) muscle wasting, (i) early stage adrenal fatigue, (j) early stage chronic fatigue syndrome, (k) psychological stress, (l) addiction, (m) arthropenia, (n) diarrheal disease, (o) cognitive deficits due to the brain's ability to utilize glucose, (p) skin aging, (q) hypertension, (r) viral infections, (s) irritable bowel syndrome, (t) storage of abdominal fat, (u) multiple sclerosis, (v) anorexia nervosa, (w) candida, (x) intestinal parasites, (y) suboptimal testosterone levels, (z) AIDS, (aa) viral hepatitis, and (ab) weight gain.

3. The method of claim 1 wherein glucans are added to said food.

4. The method of claim 3 wherein the glucans are derived from natural or hybrid mushrooms.

5. The method of claim 1 further comprising measuring hormone or cytokine levels periodically.

6. The method of claim 5 wherein said cytokine comprises any one selected from a group consisting of IL-2, IFN-gamma, TNF-alpha, IL-4, IL-5, IL-6, IL-10, and IL-13.

7. The method of claim 5 wherein said hormone comprises cortisol.

8. The method of claim 5 further comprising adjusting said dosage levels based on said measuring.

9. The method of claim 1 wherein said transfer factor in each said dosage is present at 0.05 to 50 mg per pound of human body weight.

10. The method of claim 1 wherein said lactic acid generating bacteria in each said dosage is present at 0.47 to 10 mg per pound of human body weight.

11. The method of claim 10 wherein said lactic acid generating bacteria has a live count of 2.5 billion colony forming units per ounce before inclusion into said food.

12. The method of claim 3 wherein said glucans in each said dosage are present at 0.1 to 10 mg per pound of human body weight.

13. The method of claim 1 wherein said continuing step requires between 3 days and 90 days.

14. The method of claim 3 wherein transfer factor, lactic acid generating bacteria, and glucans are consumed separately at different times within a two-day period.

15. The method of claim 1 wherein human cortisol levels exceed 15 micrograms/deciliter in the early morning or exceed 5 micrograms/deciliter at bedtime.

16. The method of claim 2 wherein weight gain is due to improvements for osteopenia, osteoporosis, bone weakness, proteolysis, or muscle wasting.

17. A method for reducing the symptoms of human disease, comprising:
combining transfer factor and lactic acid generating bacteria to create a food, wherein
said transfer factor includes polypeptides with a molecular weight below 10,000 Daltons, and
said transfer factor is derived from avian, ova or colostrum sources;
feeding the food to said human, wherein
a dosage level is chosen based on the human's weight,
feeding begins 3-90 days before measuring hormone or cytokine concentrations, and
the frequency of the feeding ranges between five times per day and once per week; and
continuing said feeding until medical or drug intervention begins.

18. The method of claim 17 wherein the hormone is cortisol.

19. A method for reducing the symptoms of an auto-immune human disease, comprising:
combining transfer factor and lactic acid generating bacteria to create a food, wherein
said transfer factor includes polypeptides with a molecular weight below 10,000 Daltons, and
said transfer factor is derived from avian, ova or colostrum sources;
feeding the food to said human, wherein
a dosage level is chosen based on the human's weight,
feeding begins 3-90 days before any medical or drug intervention, and
the frequency of the feeding ranges between five times per day and once per week; and
continuing said feeding until medical or drug intervention begins.
